(19) United States
(12) Patent Application Publication
(10) Pub. No.: US 2007/0106402 A1
(43) Pub. Date: May 10, 2007

(54) Calcium cleansing for vascular visualization

(76) Inventors: Wenli Cai, Dorchester, MA (US); Dongqing Chen, Setauket, NY (US)

Correspondence Address: F. CHAU & ASSOCIATES, LLC, 130 WOODBURY ROAD, WOODBURY, NY, 11797, US

(21) Appl. No.: 10/580,738

(22) PCT Filed: Nov. 24, 2004
(86) PCT No.: PCT/US04/39699

§ 371 Date: May 26, 2006

Related U.S. Application Data
(60) Provisional application No. 60/525,603, filed on Nov. 26, 2003.

(51) Int. Cl. G06F 17/00; A61B 6/00

(52) U.S. Cl. 700/90; 600/431

(57) ABSTRACT

A system (100) and corresponding method for vessel visualization are provided, the system having an input adapter (112, 128, 130) for receiving segmented vessel data, a processor (102) in signal communication with the input adapter, a vessel visualization unit (170) in signal communication with the processor for visualizing the vessel, and a calcium cleansing unit (180) in signal communication with the processor for removing the influences of calcium deposits from the visualized vessel; and the corresponding method including receiving segmented vessel data, visualizing a vessel in correspondence with the segmented vessel data, and cleansing calcium by removing the influences of calcium deposits from the visualized vessel.

### CROSS-REFERENCE TO RELATED APPLICATION

This application claims priority to U.S. Provisional Patent Application Ser. No. 60/525,603, filed Nov. 26, 2003, which is incorporated herein by reference in its entirety.

### BACKGROUND

The present disclosure relates to the performance of virtual examinations. More particularly, the disclosure provides a system and method for calcium cleansing in vascular visualization.

Two-dimensional (2D) visualization of human organs using medical imaging devices has been widely used for patient diagnosis. Currently available medical imaging devices include computed tomography (CT) and magnetic resonance imaging (MRI), for example. Three-dimensional (3D) images can be formed by stacking and interpolating between two-dimensional pictures produced from the scanning machines. Imaging an organ and visualizing its volume in three-dimensional space would be beneficial due to the lack of physical intrusion and the ease of data manipulation. However, the exploration of the three-dimensional volume image must be properly performed in order to fully exploit the advantages of virtually viewing an organ or vessel.

With the progress of multi-detector computerized tomography (MDCT) and increasing temporal and spatial resolution of data sets, clinical use of computerized tomographic angiography (CTA) is increasing. Vessel visualization can be quite challenging, but is needed to view vascular structures. One of the advantages of CTA over magnetic resonance angiography (MRA) is its capability to view both calcification and stenosis. However, in 3D volume rendering, and particularly in Multum In Parvo (MIP) volumes, calcium on the vessel walls may block the view of a lumen since the intensity of the calcium is higher than the intensity of the lumen. This makes it difficult to visualize the vessel lumen, or to view the stenosis in 3D. The calcium may be observed in both surface-based and MIP volume rendered images, for example.

Unfortunately, a viewer is generally unable to determine either the volume of the calcium or the stenosis caused by the calcification. For example, vessel segmentation may detect bright tubular objects on dark backgrounds. Thus, in many cases, calcium or portions with calcium are segmented as vessels. In MIP, calcium overwhelms lumen voxels in images. Therefore, what is needed is a system and method for calcium cleansing in vascular visualization, such as a virtual examination system providing data in a conventional format for analysis while, in addition, allowing an operator to easily navigate among vessels and vascular structures with and without calcium deposits.

### SUMMARY

These and other drawbacks and disadvantages of the prior art are addressed by a system and method for calcium cleansing in vascular visualization.

A system embodiment has an input adapter for receiving segmented vessel data, a processor in signal communication with the input adapter, a vessel visualization unit in signal communication with the processor for visualizing the vessel, and a calcium cleansing unit in signal communication with the processor for removing the influences of calcium deposits from the segmented vessel data.

A method embodiment includes receiving segmented vessel data, visualizing a vessel in correspondence with the segmented vessel data, and cleansing calcium by removing the influences of calcium deposits from the visualized vessel.

Another method embodiment includes thresholding to select a calcium region, filling the calcium region with a background value, and calculating a 3D Gaussian volume mask for the calcium region.

A program storage device embodiment includes program steps for receiving segmented vessel data, and cleansing calcium by removing the influences of calcium deposits from the segmented vessel data.

These and other aspects, features and advantages of the present disclosure will become apparent from the following description of exemplary embodiments, which is to be read in connection with the accompanying drawings.

### BRIEF DESCRIPTION OF THE DRAWINGS

FIG. 1 shows a schematic diagram of a system for vascular visualization with calcium cleansing in accordance with an embodiment of the present disclosure;

FIG. 2 shows graphical diagrams for vascular visualization before calcium cleansing in accordance with an embodiment of the present disclosure;

FIG. 3 shows graphical diagrams for vascular visualization after calcium cleansing in accordance with an embodiment of the present disclosure; and

FIG. 4 shows graphical diagrams for vascular visualization before and after calcium cleansing in accordance with an embodiment of the present disclosure.

### DETAILED DESCRIPTION OF EXEMPLARY EMBODIMENTS

The present disclosure provides system and method embodiments for calcium cleansing in vascular visualization. Calcium deposits may pose a greater complication in computerized tomographic angiography (CTA) than in magnetic resonance angiography (MRA), for example, but may also be an issue in other types of data, such as ultrasound, x-ray angiography (XRA) and digital subtraction angiography (DSA). A technique called calcium cleansing erases the calcium within the vessel to clearly visualize the vessel lumen. A unique advantage is the correction of the undesirable partial volume effects of calcium deposits. An exemplary embodiment of the disclosure enables physicians to diagnose stenosis with CTA.

With the progress of multi-detector computerized tomography (MDCT) and increasing temporal and spatial resolution of data sets, clinical use of CTA is increasing. Vessel visualization in the presence of calcium deposits can be quite challenging, but it is needed to analyze vascular structures.

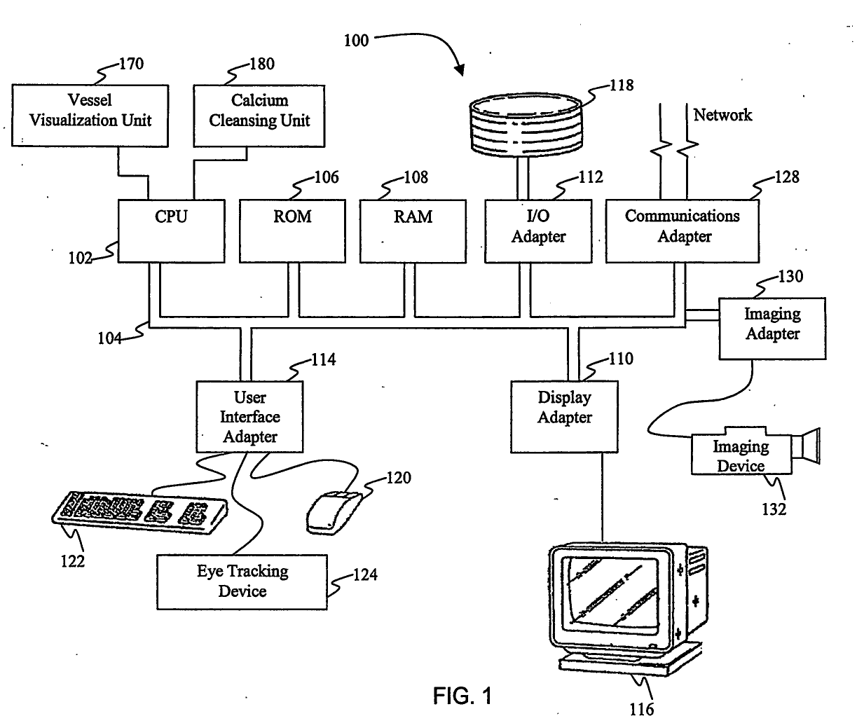

As shown in FIG. 1, a system for vessel visualization with calcium cleansing, according to an illustrative embodiment of the present disclosure, is indicated generally by the reference numeral 100. The system 100 includes at least one processor or central processing unit (CPU) 102 in signal communication with a system bus 104. A read only memory (ROM) 106, a random access memory (RAM) 108, a display adapter 110, an I/0 adapter 112, a user interface adapter 114, a communications adapter 128, and an imaging adapter 130 are also in signal communication with the system bus 104. A display unit 116 is in signal communication with the system bus 104 via the display adapter 110. A disk storage unit 118, such as, for example, a magnetic or optical disk storage unit is in signal communication with the system bus 104 via the I/O adapter 112. A mouse 120, a keyboard 122, and an eye tracking device 124 are in signal communication with the system bus 104 via the user interface adapter 114. A magnetic resonance imaging device 132 is in signal communication with the system bus 104 via the imaging adapter 130.

A vessel visualization unit 170 and a calcium cleansing unit 180 are also included in the system 100 and in signal communication with the CPU 102 and the system bus 104. While the vessel visualization unit 170 and the calcium cleansing unit 180 are illustrated as coupled to the at least one processor or CPU 102, these components are preferably embodied in computer program code stored in at least one of the memories 106, 108 and 118, wherein the computer program code is executed by the CPU 102. As will be recognized by those of ordinary skill in the pertinent art based on the teachings herein, alternate embodiments are possible, such as, for example, embodying some or all of the computer program code in registers located on the processor chip 102. Given the teachings of the disclosure provided herein, those of ordinary skill in the pertinent art will contemplate various alternate configurations and implementations of the vessel visualization unit 170 and the calcium cleansing unit 180, as well as the other elements of the system 100, while practicing within the scope and spirit of the present disclosure.

The method is demonstrated by cleansing calcium from segmented vessels, including vessels that are in close proximity to bone.

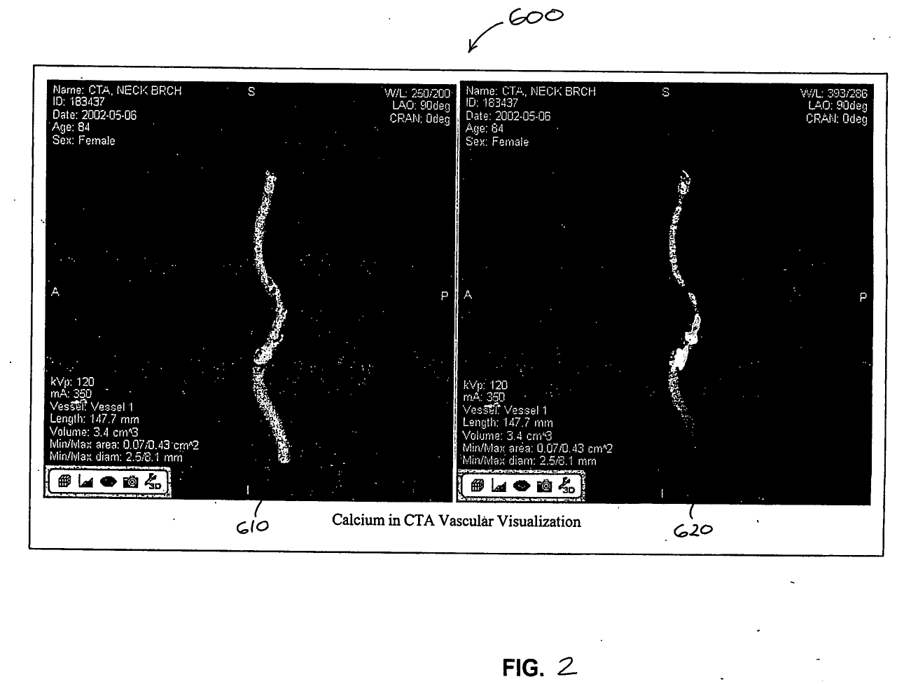

Turning now to FIG. 2, a CTA vascular visualization before calcium cleansing is indicated generally by the reference numeral 600. A visualization 610 shows a vessel segmented along with calcium deposits, while a visualization 620 shows the same calcium deposits highlighted.

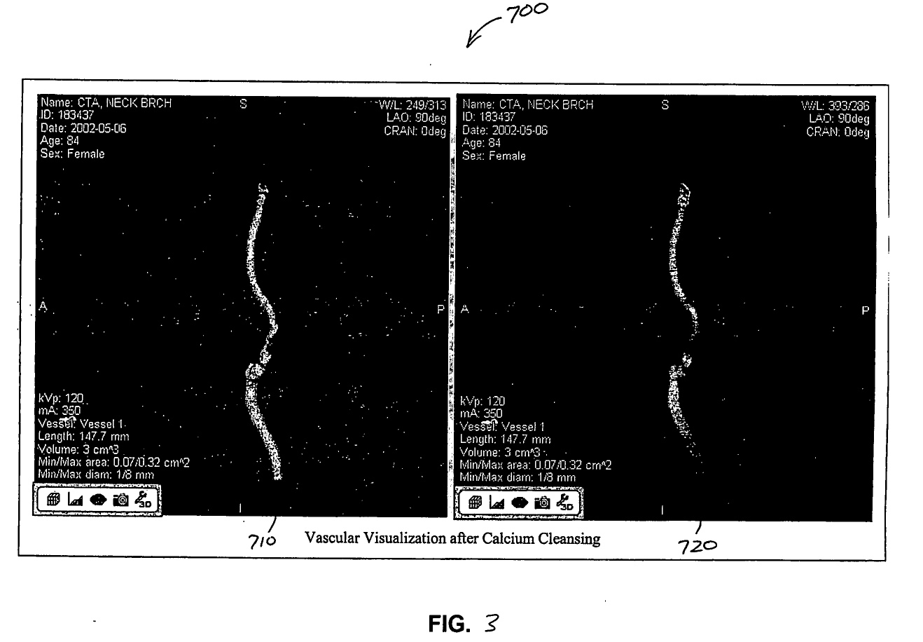

As shown in FIG. 3, a CTA vascular visualization after calcium cleansing is indicated generally by the reference numeral 700. A visualization 710 shows a vessel segmented along with calcium deposits, while a visualization 720 shows the same vessel with the calcium deposits erased.

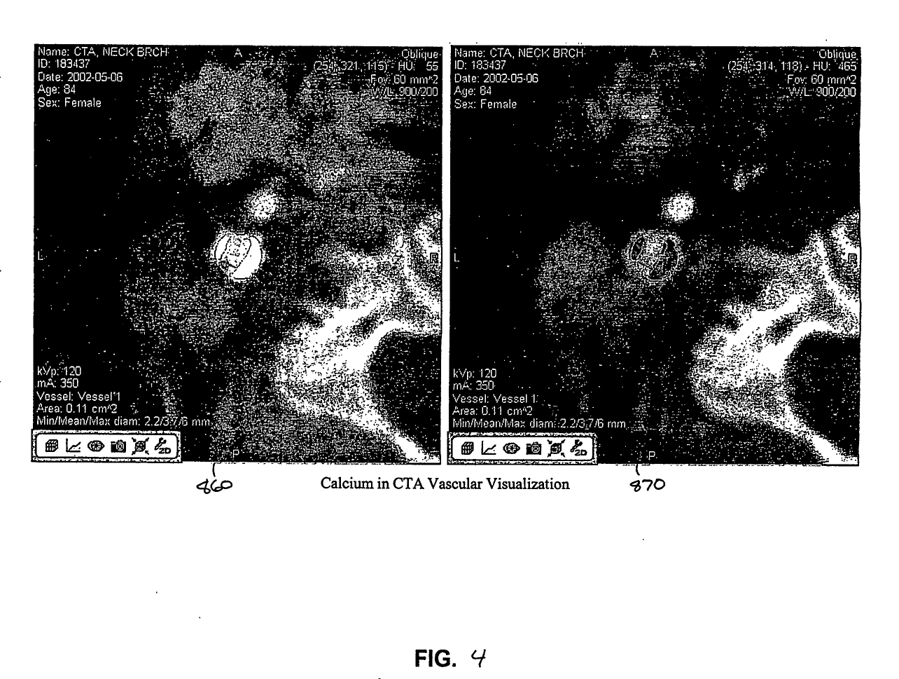

Turning to FIG. 4, 2D slice CTA vascular visualizations are indicated generally by the reference numeral 800. Here, a visualization 860 shows a vessel segmented along with calcium deposits, where the calcium deposits are highlighted. A visualization 870 shows the same vessel with the calcium deposits erased.

The calcium is found in both surface-based and MIP volume rendered images, for example. Under the prior art, a user would have no idea of the volume of calcium or the stenosis caused by the calcification. Vessel segmentation detects bright tubular objects on a dark background. Thus, in most cases, calcium or parts of calcium are segmented as vessels. In MIP, calcium overwhelms lumen voxels in images. Therefore, an exemplary embodiment of the present disclosure provides calcium cleansing in CTA vascular visualization.

In general, the intensity ranges of calcium and vessel lumen that are enhanced by contrast agents are different. For example, calcium intensity is above 500 in the exemplary embodiment, while lumen intensity is below 500. In different studies or applications, the actual intensities may vary due to the time control of contrast agents, for example. Basically, a threshold can be used to separate the calcium and vessel lumen.

It is a poor idea to only remove the calcium voxels with a thresholding. Due to partial volume effects, the voxels near the calcium have higher intensity than the lumen. Thus, the higher intensity voxels would still obscure a view to the lumen. Therefore, embodiments of the present disclosure filter the calcium region after applying the thresholding.

A preferred solution is to remove the partial volume effects by means of a Gaussian filter. Supposing that each voxel is sampled within a region with a Gaussian mask, when a voxel is changed to another value, it affects its neighbors located within the sampling radius. Thus, when the calcium regions are filled with a background value, one should remove the partial volume effects by a difference caused by filling. The radius (3.0*δ) of a Gaussian filter kernel is preferably set to δ =1.0 based on experience.

A method for calcium cleansing includes the following steps.

1) Fill the calcium region that is set by thresholding with a background value.

2) Calculate the 3D Gaussian Volume Mask (G).

3) For each calcium voxel,

(i) Calculate the difference (d) between background and original intensity;

(ii) Calculate the difference of volume mask by d*G; and

(iii) For all voxels within the volume mask, if it is not a calcium voxel, remove the corresponding difference in difference of volume mask.

Referring back to FIGS. 6, 7 and 8, a carotid CTA data set is used to illustrate the results of calcium cleansing. After calcium cleansing, a clear view of the lumen is rendered in both images 720 and 870.

Thus, preferred embodiments of the present disclosure provide a method for calcium cleansing, which removes the calcium within the vessel image to clearly visualize the vessel lumen. A unique advantage is the removal of partial volume effects of the calcium deposits, which is desired to help physicians to diagnose stenosis in CTA data sets.

The foregoing merely illustrates the principles of the disclosure. It will thus be appreciated that those skilled in the art will be able to devise numerous systems, apparatus and methods which, although not explicitly shown or described herein, embody the principles of the disclosure and are thus within the spirit and scope of the disclosure as defined by its Claims.

For example, the methods and systems described herein could be applied to virtually examine an animal, fish or inanimate object. Besides the stated uses in the medical field, applications of the technique could be used to detect the contents of sealed objects that cannot be opened. The technique could also be used inside an architectural structure such as a building or cavern and enable the operator to navigate through the structure.

These and other features and advantages of the present disclosure may be readily ascertained by one of ordinary skill in the pertinent art based on the teachings herein. It is to be understood that the teachings of the present disclosure may be implemented in various forms of hardware, software, firmware, special purpose processors, or combinations thereof.

Most preferably, the teachings of the present disclosure are implemented as a combination of hardware and software. Moreover, the software is preferably implemented as an application program tangibly embodied on a program storage unit. The application program may be uploaded to, and executed by, a machine comprising any suitable architecture. Preferably, the machine is implemented on a computer platform having hardware such as one or more central processing units (CPU), a random access memory (RAM), and input/output (I/O) interfaces. The computer platform may also include an operating system and microinstruction code. The various processes and functions described herein may be either part of the microinstruction code or part of the application program, or any combination thereof, which may be executed by a CPU., In addition, various other peripheral units may be connected to the computer platform such as an additional data storage unit and a printing unit.

It is to be further understood that, because some of the constituent system components and methods depicted in the accompanying drawings are preferably implemented in software, the actual connections between the system components or the process function blocks may differ depending upon the manner in which embodiments of the present disclosure are programmed. Given the teachings herein, one of ordinary skill in the pertinent art will be able to contemplate these and similar implementations or configurations of the present invention.

Although the illustrative embodiments have been described herein with reference to the accompanying drawings, it is to be understood that the present invention is not limited to those precise embodiments, and that various changes and modifications may be effected therein by one of ordinary skill in the pertinent art without departing from the scope or spirit of the present disclosure. All such changes and modifications are intended to be included within the scope of the present invention as set forth in the appended Claims.

What is claimed is:

1. A system for vessel visualization, comprising:
at least one input adapter for receiving segmented vessel data;
a processor in signal communication with the at least one input adapter;
a vessel visualization unit in signal communication with the processor for visualizing a vessel; and
a calcium cleansing unit in signal communication with the processor for removing the influences of calcium deposits from the visualized vessel.

2. A system as defined in claim 1, the calcium cleansing unit comprising a Gaussian filter for sampling each voxel disposed about a calcium region that has been filled with a background value.

3. A system as defined in claim 2 wherein the radius (3.0*δ) the Gaussian filter kernel is set to δ=1.0.

4. A system as defined in claim 1, further comprising a display adapter (110) in signal communication with the processor for providing a visualization of vasculature responsive to at least one of the vessel visualization unit and the calcium cleansing unit.

5. A system as defined in claim 1 wherein the vessel visualization unit provides a separation of vasculature from non-vasculature such as bones, soft tissue and calcium deposits.

6. A program storage device readable by machine, tangibly embodying a program of instructions executable by the machine to perform program steps for vessel visualization, the program steps comprising:
receiving segmented vessel data;
visualizing a vessel in correspondence with the segmented vessel data; and
cleansing calcium by removing the influences of calcium deposits from the visualized vessel.

7. A method of vessel visualization, comprising:
receiving segmented vessel data;
visualizing a vessel in correspondence with the segmented vessel data; and
cleansing calcium by removing the influences of calcium deposits from the visualized vessel.

8. A visualization method as defined in claim 7, cleansing calcium comprising:
filling a calcium region with a background value; and
sampling with a Gaussian filter each voxel disposed about the calcium region that has been filled with the background value.

9. A visualization method as defined in claim 8 wherein the radius (3.0*δ) of the Gaussian filter kernel is set to δ=1.0.

10. A visualization method as defined in claim 7, cleansing calcium comprising at least one of determining the volume of calcium and determining the stenosis caused by the calcium.

11. A visualization method as defined in claim 7 wherein calcium or areas of calcium are segmented as vessels in the segmented vessel data.

12. A visualization method as defined in claim 7, cleansing calcium comprising:
removing the calcium voxels with a thresholding; and
correcting partial volume effects, where the voxels near the calcium have higher intensity than the lumen, by filtering about the calcium region after thresholding.

13. A visualization method as defined in claim 12, correcting partial volume effects comprising:
applying a Gaussian filter such that each voxel is sampled within a region with a Gaussian mask; and
removing the partial volume effects by a difference caused by filling calcium regions with a background value.

14. A method for calcium cleansing, comprising:
thresholding to select a calcium region;
filling the calcium region with a background value; and
calculating a 3D Gaussian Volume Mask (“G”).

15. A cleansing method as defined in claim 14, further comprising:
calculating the difference (“d”) between background and original intensity values for each voxel disposed about the calcium region;
calculating the difference of volume mask by d*G for each voxel disposed about the calcium region; and
for all voxels within the volume mask that are not calcium voxels, removing the corresponding difference in the volume mask.

16. A cleansing method as defined in claim 14, further comprising removing the calcium within a computerized tomographic angiography (CTA) data set corresponding to vessel data to clearly visualize the vessel lumen, including the removal of partial volume effects of the calcium deposits.

17. A cleansing method as defined in claim 14, further comprising:
receiving segmented vessel data; and
visualizing a vessel in response to the segmented vessel data and the Gaussian volume mask by removing the influences of calcium deposits from the visualized vessel.

18. A cleansing method as defined in claim 17, further comprising:
calculating the difference (“d”) between background and original intensity values for each voxel disposed about the calcium region;
calculating the difference of volume mask by d*G for each voxel disposed about the calcium region; and
for all voxels within the volume mask that are not calcium voxels, removing the corresponding difference in the volume mask.

19. A cleansing method as defined in claim 17, the method further comprising:
removing the calcium within the segmented vessel data to clearly visualize the vessel lumen; and
correcting partial volume effects of the calcium.

20. A cleansing method as defined in claim 19, correcting partial volume effects comprising:
applying a Gaussian filter such that each voxel is sampled within a region with a Gaussian mask; and
removing the partial volume effects by a difference caused by filling calcium regions with a background value.

21. A method as defined in claim 17 wherein the segmented vessel data is indicative of a computerized tomographic angiography (CTA) data set.
